(19) United States
(12) Patent Application Publication
(10) Pub. No.: US 2005/0036955 A1
(43) Pub. Date: Feb. 17, 2005

(54) Bioresorbable tooth extraction socket dressing

(76) Inventor: Michael D. DeGould, Rockford, IL (US)

Correspondence Address: QUARLES & BRADY LLP, 411 E. WISCONSIN AVENUE, SUITE 2040, MILWAUKEE, WI, 53202-4497, US

(21) Appl. No.: 10/640,366

(22) Filed: Aug. 13, 2003

(51) Int. Cl. A61K 7/16; A61K 7/20

(52) U.S. Cl. 424/53; 433/217.1

(57) ABSTRACT

A moldable, bioresorbable, biocompatible, non-allergenic crosslinked collagen derivative dressing for the prevention of post extraction alveolar osteitis (dry socket) pain is disclosed along with methods for use of the gel. The dressing is placed at the time of surgery acting as a bone covering obtundant and physiologic scaffolding for the conduction of normal alveolar bone healing sequence of fibroblast ingrowth, blood vessel formation, and reossification of the extraction site defect. In one form, the dressing is a flowable, moldable, biocompatible, bioresorbable dressing prepared by reacting (i) a collagen derivative, such as gelatin or atelocollagen, and (ii) a non-cytotoxic crosslinking agent.

### CROSS-REFERENCES TO RELATED APPLICATIONS

Not Applicable.

### STATEMENT REGARDING FEDERALLY SPONSORED RESEARCH

Not Applicable.

### BACKGROUND OF THE INVENTION

1. Field of the Invention

This invention relates in general to surgical dressings, and in particular to a dressing for the prevention of alveolar osteitis and pain following the removal of a tooth or jaw cyst, and especially relates to a method of using non-cytotoxic crosslinking agents for the manufacture of a nonallergenic, biocompatible, bioresorbable bony wound dressing.

2. Description of the Related Art

Development of painful non-healing of extraction site or cystic cavity defect in the jaws commonly known as dry socket (alveolar osteitis) is a well known phenomena, with an incidence rate of between 10 to 40%. Symptoms include severe, unrelenting pain that begins within 48 to 72 hours after surgery and can persist for up to 30 days. This condition is unresponsive to oral analgesics and requires a second treatment intervention for pain relief. A dry socket can occur with the removal of any tooth, but is particularly associated with the removal of mandibular third molars (wisdom teeth). The etiology is believed to be non-formation of extraction site clot or premature loss of clot resulting in painful exposed bony extraction site surfaces. Regardless of cause, dry socket remains a prevalent and painful condition.

Current treatment of the condition requires multiple patient visits for the packing of the socket with cotton ribbon gauze impregnated extemporaneously with a variety of medicaments including eugenol, aspirin, or petroleum jelly. These packings are not sterile, usually require local anesthetic injection for placement, and have a brief duration of analgesic effect requiring their replacement every 24 to 48 hours.

Alternative materials and methods for preventing and/or treating dry socket have been proposed. U.S. Pat. No. 5,297,563 describes mechanical barriers for tissue regeneration in a bony deficit such as that remaining after tooth extraction. The barrier may be formed from gelatin and it is stated as preventing dry socket. U.S. Pat. No. 5,006,071 describes a method for the prevention of alveolar osteitis following tooth extraction. The method involves placing in an extraction cavity a gelatin sponge with absorbed plaster of Paris. As the cavity heals, the dressing is absorbed and permits regeneration of bone in the socket.

Other oral wound dressings are also known in the art, For example, U.S. Pat. No. 6,458,380 describes a dressing and method for treating wounds such as a wound in an oral cavity. The dressing may include, among other things, a polymer component comprising collagen or gelatin. Materials for guided tissue regeneration in dental applications are also known. Collagen membranes are used in dentistry for guided bony tissue regeneration around teeth and dental implant use. For example, U.S. Pat. Nos. 6,346,515 and 5,955,438 show that collagen matrix crosslinked with certain reducing sugars may be used for guided tissue regeneration in dental applications. Materials for the treatment of periodontal defects are also known. U.S. Pat. No. 6,123,957 describes the use of collagen in a method for treating periodontal disease wherein the treatment composition may be inserted in a periodontal defect.

Conventional collagen derivatives such as gelatin lack thermal stability which results in rapid dissipation of the material from a wound, limiting its utility as a wound dressing. For example, conventional gelatin melts at about 28-32 degrees Celsius depending on protein concentration. This melting is reversible with the solution converting back to the gel state upon lowering the temperature. Because of the disadvantages of unmodified gelatin in wound dressings, it has been proposed to crosslink gelatin to improve its physical properties. For instance, glutaraldehyde has been proposed as a gelatin crosslinker. However, numerous patents recognize the cytotoxicity of glutaraldehyde and alternative gelatin crosslinkers have been proposed. See, for example, U.S. Pat. Nos. 5,412,076, 5,314,874, 5,147,344, 4,971,954, 4,703,108, 4,592,864 and European patent applications EP 702959 and EP 268921.

Another factor limiting the medical application of collagen products is the antigenicity (foreign body reaction) associated with the terminal telopeptide region of the collagen molecule. It is known to those skilled in the art that these terminal telopeptide regions can be removed by the enzymatic action of pepsin, rendering the nonallergenic atelocollagen material. See, for example U.S. Pat. No. 5,314,874 and European patent application EP 268921.

Therefore, there remains a need for a sterile, biocompatible, non-cytotoxic wound dressing placed at the time of surgery for the prevention of dry socket. Such a dressing would eliminate slow, painful, non-healing of an extraction site or bony cystic defects by promoting tissue growth and healing, and would not require removal.

### SUMMARY OF THE INVENTION

The foregoing needs are met by a method according to the invention for the prevention of alveolar osteitis and pain following tooth extraction or jaw cyst removal. The method includes the steps of (a) filling an oral cavity remaining after tooth extraction or jaw cyst removal with a flowable, moldable, biocompatible, bioresorbable dressing prepared by reacting (i) a collagen derivative and (ii) a non-cytotoxic crosslinking agent, and (b) enclosing the dressing in the cavity.

In one form, the dressing is a flowable, moldable, biocompatible, bioresorbable dressing prepared by reacting (i) gelatin and (ii) a non-cytotoxic crosslinking agent. In another form, the dressing is a flowable, moldable, biocompatible, bioresorbable dressing prepared by reacting (i) atelocollagen, and (ii) a non-cytotoxic crosslinking agent.

In another aspect, the invention provides a kit for use in the method for the prevention of alveolar osteitis and pain following tooth extraction or jaw cyst removal. The kit is a sterile package having a syringe loaded with the wound dressing according to the invention.

It is therefore an advantage of the present invention to provide a method for the prevention of alveolar osteitis and pain following tooth extraction or jaw cyst removal wherein a dressing is placed in an oral cavity remaining after tooth extraction or jaw cyst removal to act as a bone covering obtundant and physiologic scaffolding for the conduction of normal alveolar bone healing sequence of fibroblast ingrowth, blood vessel formation, and reossification of the extraction site defect.

It is another advantage of the present invention to provide a dressing for the prevention of alveolar osteitis and pain following tooth extraction or jaw cyst removal wherein the dressing is physically stable and non-cytotoxic when placed in an oral cavity remaining after tooth extraction or jaw cyst removal.

These and other features, aspects, and advantages of the present invention will become better understood upon consideration of the following detailed description and appended claims.

### DETAILED DESCRIPTION OF THE INVENTION

The present invention is directed to a method for the prevention of alveolar osteitis and pain following tooth extraction or jaw cyst removal. The method includes the steps of (a) filling an oral cavity remaining after tooth extraction or jaw cyst removal with a flowable, moldable, biocompatible, bioresorbable dressing prepared by reacting (i) a collagen derivative and (ii) a non-cytotoxic crosslinking agent, and (b) enclosing the dressing in the cavity. Typically, the dressing is placed in the oral cavity with a medical syringe, and is enclosed in the oral cavity by suturing tissue above the placed dressing.

The present invention is also directed to a wound dressing that may be placed in an oral cavity for the prevention of alveolar osteitis and pain following tooth extraction or jaw cyst removal. The dressing is a flowable, moldable, biocompatible, bioresorbable dressing prepared by reacting (i) a collagen derivative and (ii) a non-cytotoxic crosslinking agent. As used herein, a collagen derivative is a substance which is obtained by chemically or physically altering naturally occurring collagen. For example, one preferred collagen derivative, gelatin, can be obtained by boiling collagen in water. Another preferred collagen derivative, atelocollagen, can be obtained by digesting the telopeptide moiety in collagen with an enzyme such as pepsin.

As used herein, a “non-cytotoxic” crosslinking agent is a crosslinking agent that is not cytotoxic by itself and/or that does not render the collagen derivative cytotoxic after the crosslinking reaction. Suitable non-cytotoxic crosslinking agents include, without limitation, compounds containing metal cations, peroxides, and mixtures thereof. Examples of compounds containing metal cations include compounds having Cu²⁺, Fe²⁺ and Al³⁺ cations. One preferred non-cytotoxic crosslinking agent is a compound including copper cations such as cupric chloride. Another preferred non-cytotoxic crosslinking agent is hydrogen peroxide.

As used herein, a “biocompatible” material is one which stimulates only a mild, often transient, implantation response, as opposed to a severe or escalating response. As used herein, a “bioresorbable” material is one that breaks down over a finite period of time due to the chemical/biological action of the body. As used herein, a material is “moldable” if the material can conform to and fill a cavity. As used herein, a material is “syringable” if the material may be delivered to a site by way of a medical syringe.

One or more bioactive agents may also be incorporated into the dressing. The bioactive agent or bioactive agents are selected depending on the physiological effect desired. A “bioactive agent” as used herein includes, without limitation, physiologically or pharmacologically active substances that act locally or systemically in the body. A bioactive agent is a substance used for the treatment, prevention, diagnosis, cure or mitigation of disease or illness, or a substance which affects the structure or function of the body or which becomes biologically active or more active after it has been placed in a predetermined physiological environment. Bioactive agents include, without limitation, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, extracellular matrix and/or its individual components, mammalian cells, genetically engineered cells, pharmaceuticals, and therapeutics.

A dressing according to the invention is a syringable collagen derivative gel having the consistency of a viscous paste that is placed at the time of surgery to substantially fill and assume the shape of the oral bone cavity. The dressing is allowed to remain in the bony cavity for the prevention of pain and promotion of rapid healing. The dressing is absorbed while conducting cell growth resulting in uninterrupted regeneration of bone in the cavity. The dressing is bioresorbable by the human body, and has high viscosity for displacing blood from the cavity and allowing bony adherence. The dressing is slightly resilient for aiding in filling the cavity, and is moldable for reducing wound pressure and for aiding in filling the cavity. The dressing is crosslinked in order to stabilize the dressing increasing its residence time in the oral cavity. The crosslinking agents used in producing the dressing are non-cytotoxic.

The wound dressing may be placed in an oral cavity remaining after tooth extraction as follows. After surgical removal of a tooth (e.g., a wisdom tooth), a cavity is left in the mandible. An incised portion of mucosal tissue, typically a flap, may partially cover the cavity. The wound dressing is placed in the cavity using a carrying instrument or syringe. This placement typically occurs contemporaneously with the surgical procedure responsible for surgical removal of the tooth, but may occur at another time in some cases. The mucosal flap tissue may then be sutured over the wound dressing thereby enclosing the dressing in the cavity.

### EXAMPLE

This Example has been presented in order to further illustrate the invention and is not intended to limit the invention in any way.

Thermal stabilization of atelocollagen into a slowly soluble gel at body temperature can be achieved using non-cytotoxic cross linking agents by the following method. There are no restrictions on the atelocollagen source used in the present invention. Skin, bone, tendon or fascia from cows, pigs or chickens may be used and types I, III, and V atelocollagen are appropriate for use.

Prepare an atelocollagen solution with HCL pH 2-3 containing 0.3% w/v atelocollagen in an ice bath. Add sodium phosphate buffer titrated to a pH of 7-8 in a final concentration of 0.27% atelocollagen and 30 mM sodium phosphate Buffer. Then add crosslinking agents cupric chloride 0.1 mM and a hydrogen peroxide 0.3% and allow to gel at 20-45 Celsius for 1 minute to 24 hours, whereupon 10 mM EDTA as a chelator is added producing the dressing of the present invention. Final viscosity of the preparation can be modified by variance of the atelocollagen or crosslinking agent concentration. The material is then packaged in 5 ml. curve tipped syringes which are sealed in cellophane and sterilized by exposure to 2.5 mRads of gamma radiation.

Thus, it can be seen that there has been provided a dry socket preventative dressing that is sterile, stable, biocompatible, and can be efficiently placed at the time of surgery.

Although the present invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A method for the prevention of alveolar osteitis and pain following tooth extraction or jaw cyst removal, the method comprising:
(a) filling an oral cavity remaining after tooth extraction or jaw cyst removal with a flowable, moldable, biocompatible, bioresorbable dressing prepared by reacting (i) a collagen derivative and (ii) a non-cytotoxic crosslinking agent; and
(b) enclosing the dressing in the cavity.

2. The method of claim 1 wherein:
the collagen derivative is gelatin.

3. The method of claim 1 wherein:
the collagen derivative is atelocollagen.

4. The method of claim 1 wherein:
step (b) comprises enclosing the dressing in the cavity by suturing tissue above the dressing.

5. The method of claim 1 wherein:
step (a) comprises filling the dressing in the oral cavity with a medical syringe.

6. The method of claim 1 wherein:
the crosslinking agent is selected from compounds containing metal cations, peroxides, and mixtures thereof.

7. The method of claim 1 wherein:
the crosslinking agent is selected from compounds containing copper cations, hydrogen peroxide, and mixtures thereof.

8. The method of claim 1 wherein:
the melting point of the dressing is above 38 degrees Celsius.

9. The method of claim 1 wherein:
the collagen derivative is gelatin, and the crosslinking agent is selected from compounds containing metal cations, peroxides, and mixtures thereof.

10. The method of claim 1 wherein:
the collagen derivative is atelocollagen, and
the crosslinking agent is selected from compounds containing metal cations, peroxides, and mixtures thereof.

11. The method of claim 1 wherein:
the dressing is a gel.

12. A wound dressing that may be placed in an oral cavity for the prevention of alveolar osteitis and pain following tooth extraction or jaw cyst removal, the dressing comprising:
a flowable, moldable, biocompatible, bioresorbable dressing prepared by reacting (i) a collagen derivative and (ii) a non-cytotoxic crosslinking agent.

13. The dressing claim 12 wherein:
the collagen derivative is gelatin.

14. The dressing claim 12 wherein:
the collagen derivative is atelocollagen.

15. The wound dressing of claim 12 wherein:
the dressing is syringable.

16. The wound dressing of claim 12 wherein:
the crosslinking agent is selected from compounds containing metal cations, peroxides, and mixtures thereof.

17. The wound dressing of claim 12 wherein:
the crosslinking agent is selected from compounds containing copper cations, hydrogen peroxide, and mixtures thereof.

18. The wound dressing of claim 12 wherein:
the melting point of the dressing is above 38 degrees Celsius.

19. The wound dressing of claim 12 wherein:
the collagen derivative is gelatin, and the crosslinking agent is selected from compounds containing metal cations, peroxides, and mixtures thereof.

20. The wound dressing of claim 12 wherein:
the collagen derivative is atelocollagen, and the crosslinking agent is selected from compounds containing metal cations, peroxides, and mixtures thereof.

21. The wound dressing of claim 12 wherein:
the dressing is a gel.

22. A kit for use in a method for the prevention of alveolar osteitis and pain following tooth extraction or jaw cyst removal in which an oral cavity remaining after tooth extraction or jaw cyst removal is filled with a dressing, the kit comprising:
a syringe loaded with the wound dressing of claim 12.

23. A kit for use in a method for the prevention of alveolar osteitis and pain following tooth extraction or jaw cyst removal in which an oral cavity remaining after tooth extraction or jaw cyst removal is filled with a dressing, the kit comprising:
a syringe loaded with the wound dressing of claim 13.

24. A kit for use in a method for the prevention of alveolar osteitis and pain following tooth extraction or jaw cyst removal in which an oral cavity remaining after tooth extraction or jaw cyst removal is filled with a dressing, the kit comprising:
a syringe loaded with the wound dressing of claim 14.
